(19) United States
(12) Patent Application Publication
(10) Pub. No.: US 2002/0076761 A1
(43) Pub. Date: Jun. 20, 2002

(54) Secreted human proteins

(76) Inventors: Jaime Escobedo, Alamo, CA (US); Pablo Dominguez Garcia, San Francisco, CA (US); Qianjin Hu, Castro Valley, CA (US); Srinivas Kothakota, Santa Monica, CA (US); Lewis T. Williams, Mill Valley, CA (US)

Correspondence Address: Chiron Corporation, Intellectual Property R338, P.O. Box 8097, Emeryville, CA, 94662-8097, US

(21) Appl. No.: 09/935,390

(22) Filed: Aug. 22, 2001

Related U.S. Application Data
(63) Continuation of application No. 08/988,671, filed on Dec. 11, 1997, now abandoned.
(60) Provisional application No. 60/032,757, filed on Dec. 11, 1996.

(51) Int. Cl. C12Q 1/68; C07H 21/04; C12N 9/00; C12P 21/02; C12N 5/06; C07K 14/435

(52) U.S. Cl. 435/69.1; 435/183; 435/320.1; 435/325; 435/6; 530/350; 536/23.5

(57) ABSTRACT

Secreted proteins can be identified using a method which exploits the ability of microsomes to modify proteins post-translationally. Nineteen human secreted proteins and full-length cDNA sequences encoding the proteins have been identified using this method. The proteins and cDNA sequences can be used, inter alia, for targeting other proteins to the membrane or extracellular milieu.

This application claims the benefit of copending provisional application Serial No. 60/032,757, filed Dec. 11, 1996, which is incorporated herein by reference.

### TECHNICAL AREA OF THE INVENTION

The invention relates to the area of proteins. More particularly, the invention relates to human secreted proteins.

### BACKGROUND OF THE INVENTION

Secreted proteins include such important proteins as growth factors, cytokines and their receptors, extracellular matrix proteins, and proteases. Nucleotide sequences encoding these proteins can be used to detect disease states in which such proteins are implicated and to develop therapeutics for such diseases. Thus, there is a need in the art for methods of identifying secreted proteins and the nucleotide sequences which encode them.

### SUMMARY OF THE INVENTION

It is an object of the invention to provide an isolated and purified human protein.

It is yet another object of the invention to provide a fusion protein.

It is still another object of the invention to provide a preparation of antibodies.

It is even another object of the invention to provide an isolated and purified subgenomic polynucleotide.

It is yet another object of the invention to provide an isolated gene.

It is a further object of the invention to provide a DNA construct for expressing all or a portion of a human protein.

It is still another object of the invention to provide a host cell comprising a DNA construct.

It is another object of the invention to provide a homologously recombinant cell.

It is even another object of the invention to provide a method of producing a human protein.

It is another object of the invention to provide a method of identifying a secreted polypeptide which is modified by rough microsomes.

These and other objects of the invention are provided by one or more of the embodiments described below.

One embodiment of the invention provides an isolated and purified human protein. The isolated and purified human protein has an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

Another embodiment of the invention provides an isolated and purified human protein having an amino acid sequence which is at least 85% identical to an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

Still another embodiment of the invention provides a polypeptide comprising at least 6 contiguous amino acids of an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

Even another embodiment of the invention provides a fusion protein. The fusion protein comprises a first protein segment and a second protein segment fused together by means of a peptide bond. The first protein segment consists of at least 6 contiguous amino acids selected from the group consisting of the amino acid sequences shown in SEQ ID Nos: 20, 21, 22, 23, 24, 25, 26, 27, 28, 29, 30, 31, 32, 33, 34, 35, 36, 37, and 38.

Yet another embodiment of the invention provides a preparation of antibodies. The antibodies specifically bind to a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

Even another embodiment of the invention provides an isolated and purified subgenomic polynucleotide. The isolated and purified subgenomic polynucleotide has a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

Yet another embodiment of the invention provides an isolated and purified subgenomic polynucleotide consisting of at least 10 contiguous nucleotides selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

Still another embodiment of the invention provides an isolated gene. The isolated gene corresponds to a cDNA sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

Another embodiment of the invention provides a DNA construct for expressing all or a portion of a human protein. The DNA construct comprises a promoter and a polynucleotide segment. The polynucleotide segment encodes at least 6 contiguous amino acids of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38. The polynucleotide segment is located downstream from the promoter. Transcription of the polynucleotide segment initiates at the promoter.

Even another embodiment of the invention provides a host cell comprising a DNA construct. The DNA construct comprises a promoter and a polynucleotide segment. The polynucleotide segment encodes at least 6 contiguous amino acids of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38. The polynucleotide segment is located downstream from the promoter. Transcription of the polynucleotide segment initiates at the promoter.

Still another embodiment of the invention provides a homologously recombinant cell having incorporated therein a new transcription initiation unit. The transcription initiation unit comprises in 5′ to 3′ order an exogenous regulatory sequence, an exogenous exon, and a splice donor site. The transcription initiation unit is located upstream to a coding sequence of a gene. The gene comprises a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19. The exogenous regulatory sequence controls transcription of the coding sequence of the gene.

Yet another embodiment of the invention provides a method of producing a human protein. A culture of a cell is grown. The cell comprises a DNA construct. The DNA construct comprises a promoter and a polynucleotide segment. The polynucleotide segment encodes at least 6 contiguous amino acids of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38. The polynucleotide segment is located downstream from the promoter. Transcription of the polynucleotide segment initiates at the promoter. The protein is purified from the culture.

Even another embodiment of the invention provides a method of producing a human protein. A culture of a cell is grown. The cell comprises a new transcription initiation unit. The transcription initiation unit comprises in 5′ to 3′ order an exogenous regulatory sequence, an exogenous exon, and a splice donor site. The transcription initiation unit is located upstream to a coding sequence of a gene. The gene comprises a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19. The exogenous regulatory sequence controls transcription of the coding sequence of the gene. The protein is purified from the culture.

Another embodiment of the invention provides a method of identifying a secreted polypeptide which is modified by rough microsomes. A population of cDNA molecules is transcribed in vitro whereby a population of cRNA molecules is formed. A first portion of the population of cRNA molecules is translated in vitro in the absence of rough microsomes whereby a first population of polypeptides is formed. A second portion of the population of cRNA molecules is translated in vitro in the presence of rough microsomes whereby a second population of polypeptides is formed. The first population of polypeptides is compared with the second population of polypeptides. Polypeptide members of the second population which have been modified by the rough microsomes are detected.

The present invention thus provides the art with a method for identifying secreted proteins or polypeptides, the amino acid sequences of nineteen novel human secreted proteins, and the nucleotide sequences which encode these proteins. The invention can be used to, inter alia, to produce secreted proteins for therapeutic and diagnostic purposes.

### DETAILED DESCRIPTION OF THE PREFERRED EMBODIMENTS

The inventors have discovered a method for identifying secreted proteins or polypeptides. Secreted proteins or polypeptides include soluble proteins which can be transported across a membrane, such as a cell membrane, nuclear membrane, or membrane of the endoplasmic reticulum, as well as proteins which can be partially secreted from a cell, such as membrane-bound receptors.

Secreted proteins can contain a signal (or secretion leader) sequence, located at the N-terminus and including at least several hydrophobic amino acids, such as phenylalanine, methionine, leucine, valine, or tryptophan. Non-hydrophobic amino acids can also be included in the signal sequence. Signal sequences are described in von Heijne, J. Mol. Biol. 184:99-105 (1985) and Kaiser and Botstein, Mol. Cell. Biol. 6:2382-2391 (1986). Secreted proteins can also be glycosylated by post-translational modification. The presence of a signal sequence or the presence of glycosylation or both indicate that a particular protein is a secreted protein.

In order to identify secreted proteins or polypeptides, the method of the invention exploits properties of microsomes, which are the closed vesicles that result from fragmentation of endoplasmic reticulum. Microsomes can be rough or smooth, depending on whether the endoplasmic reticulum from which they were derived is studded with ribosomes, Microsomes, particularly rough microsomes, have the ability to perform post-translational modifications, such as glycosylation and cleavage of signal sequences from proteins or polypeptides.

To identify secreted proteins, a population of complementary DNA (cDNA) molecules is transcribed in vitro to synthesize a population of complementary RNA (cRNA) molecules. The cDNA molecules can be synthesized by reverse transcription of mRNA molecules isolated from a particular cell or tissue type or organism using, for example, a commercially available reverse transcriptase enzyme. Alternatively, the reverse transcription reaction to form cDNA molecules can be conducted on total RNA, without a preliminary purification of mRNA.

Any organism, such as a bacterium, plant, invertebrate, or vertebrate organism, can be used as a source of RNA. Particularly preferred sources of RNA are mammals, most preferably humans. Tissues, such as liver, brain, kidney, spleen, pancreas, or muscle, can be used as a source of RNA. Individual cell types, either primary cells or members of established cell lines, such as HeLa, CHO, PC12, P19, BHK, COS, or HepG2, are suitable sources of RNA. Tissues or primary cells isolated from organisms at a particular stage in development can be used as RNA sources. Stem cells, such as hematopoietic, neuronal, and embryonic stem cells, can also be used as a source of RNA.

Total RNA or mRNA can be isolated using methods known in the art. Such methods are described, inter alia, in Sambrook et al., MOLECULAR CLONING, A LABORATORY MANUAL (2d ed., Cold Spring Harbor Press, N.Y., 1989), and Ausubel et al., CURRENT PROTOCOLS IN MOLECULAR BIOLOGY (Greene Publishing Associates and John Wiley & Sons, N.Y., 1994). Techniques for RNA isolation can be tailored for a particular organism or cell type, as is known in the art.

Complementary DNA can optionally be obtained from a cDNA library. The cDNA library can be derived from the genome of any organism of interest, particularly a mammal or a human. Tissue- or cell type-specific cDNA libraries can also be used as a source of cDNA.

Transcription of cDNA molecules in vitro to form cRNA molecules can be carried out using any methods known in the art. These methods include, for example, placing cDNA into a cloning vector containing a promoter, such as an SP6, T7, or T3 polymerase promoter, and transcribing the cDNA using the appropriate polymerase. A variety of commercial kits are available for this purpose.

A first portion of the population of cRNA molecules can be translated in vitro, in the absence of rough microsomes, to form a first population of polypeptides which have not been post-translationally modified. A second portion of the population of cRNA molecules can be translated in vitro in the presence of rough microsomes. Under the conditions of the in vitro translation reaction, rough microsomes can cleave signal sequences from those polypeptides which comprise such sequences. Under the same conditions, rough microsomes can also glycosylate those polypeptides which contain glycosylation sites.

Methods of in vitro translation are those which are known in the art, such as translation in a reticulocyte lysate system, particularly a rabbit reticulocyte lysate. Reticulocyte lysate systems can be assembled in the laboratory or purchased commercially in kit form.

Microsomes can be prepared by disruption of tissues or cells by homogenization, as is known in the art. If desired, rough and smooth microsomes can be separated using well-known techniques, such as sucrose density gradient sedimentation. Microsomes are also available commercially, for example, such as the canine pancreatic microsomes available from Promega Corp., Madison, Wis.

The first population of polypeptides can then be compared with the second population of polypeptides. This comparison can be by means of, for example, one- or two-dimensional polyacrylamide gel electrophoresis, as is known in the art. Polypeptides separated in the gels can be detected by any means known in the art, such as staining with copper, silver, Coomassie Brilliant Blue, amido black, fast green FCF, Ponceau S, or a chromophoric label. Separated proteins can also be visualized using radioactive, chemiluminescent, fluorescent, or enzymatic tags incorporated into the proteins before separation.

The gels can be dried or the proteins can be transferred to membranes, such as polyvinylidene difluoride membranes. Either the gels or membranes themselves or photographs of the gels or membranes can be compared by eye. Alternatively, the gels or membranes can be scanned, for example, with a densitometer and analyzed with the aid of a computer.

Polypeptide members of the second population of polypeptides, which have been modified by the rough microsomes, can be detected by any means available in the art. For example, a shift in the position of a polypeptide band can be observed, indicating an increase in molecular weight of a member of the second population compared with the corresponding polypeptide member of the first population. Such an increase in molecular weight indicates that the polypeptide member of the second population was glycosylated by the rough microsomes.

A shift in the position of a polypeptide band indicating a decrease in molecular weight of a member of the second population compared with the corresponding polypeptide member of the first population can also be observed. This decrease in molecular weight indicates that the polypeptide member of the second population contained a signal sequence which was cleaved by the rough microsomes.

Polypeptides which are modified by the rough microsomes are identified as secreted polypeptides. Optionally, quantities of cDNA molecules which encode secreted polypeptides can be obtained. Molecules of cDNA which encode polypeptides which are post-translationally modified by the rough microsomes can be placed into suitable vectors using standard recombinant DNA techniques and used to transform host cells. Many vectors are available for this purpose, such as retroviral or adenoviral vectors and bacteriophage, as described below.

Vectors comprising cDNA which encode secreted polypeptides can be introduced into host cells using techniques available in the art. These techniques include, but are not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, and calcium phosphate-mediated transfection.

The host cells can be any host cells which are capable of propagating cDNA molecules. A variety of host cells, for example immortalized cell lines such as HeLa, CHO, or HEK, are available for this purpose.

Transformed host cells can be diluted serially and cultured to form individual colonies. Methods of culturing host cells and the media suitable for each host cell type are well known in the art. Preferably, each colony originates from a single transformed host cell. Separate preparations of cDNA from each colony can be prepared, as described above, and transcribed in vitro to form cRNA. The cRNA can be transcribed to form secreted polypeptides, which can be purified as is known in the art. If the preparation of secreted polypeptides from a colony contains more than one species of polypeptide, the steps described above can be repeated until a colony is obtained which contains cDNA encoding only a single species of polypeptide.

Complementary DNA molecules which encode secreted proteins can be sequenced using standard nucleotide sequencing techniques. The sequence of each cDNA molecule can be compared with known sequences in a database to determine whether the clone encodes a known or a novel secreted protein.

The inventors have used the method of the invention to identify nineteen novel human secreted proteins. Amino acid sequences for these nineteen human secreted proteins are disclosed in SEQ ID Nos: 20, 21, 22, 23, 24, 25, 26, 27, 28, 29, 30, 31, 32, 33, 34, 35, 36, 37, and 38. Nucleotide sequences which encode the proteins are disclosed in SEQ ID NOs: 1, 2, 3, 4, 5, 6, 7, 8, 9, 10, 11, 12, 13, 14, 15, 16, 17, 18, and 19, respectively.

Clones containing the cDNAs of the secreted proteins were deposited on Dec. 11, 1997, with the ATCC. Individual bacterial cells (E. coli) in this composite deposit contain one or more of the polynucleotides encoding the secreted proteins of the invention and can be retrieved using an oligonucleotide probe designed from the sequence for that particular polynucleotide, as provided herein. Each polynucleotide can be removed from the vector by performing an EcoRI/NotI digestion (5′ site, EcoRI; 3′ site, NotI). The deposit submitted to the ATCC has been designated SECP120997. The nucleotide sequences of these deposits and the amino acid sequences they encode are controlling in the event of a discrepancy between the amino acid and nucleotide sequences disclosed herein and those contained in the deposits.

A purified and isolated subgenomic polynucleotide of the present invention comprises at least 10, 12, 15, 18, 20, 25, 30, 35, 40, 45, or 50 contiguous nucleotides selected from the nucleotide sequences shown in SEQ ID NOs: 1, 2, 3, 4, 5, 6, 7, 8, 9, 10, 11, 12, 13, 14, 15, 16, 17, 18, and 19. The isolated and purified subgenomic polynucleotides can comprise an entire nucleotide sequence selected from the nucleotide sequences shown in SEQ ID NOs: 1, 2, 3, 4, 5, 6, 7, 8, 9, 10, 11, 12, 13, 14, 15, 16, 17, 18, and 19.

Subgenomic polynucleotides contain less than a whole chromosome and are preferably intron-free. Polynucleotides of the invention can be isolated and purified free from other nucleotide sequences by standard nucleic acid purification techniques, using restriction enzymes and probes to isolate fragments comprising the coding sequences.

Isolated genes corresponding to the cDNA sequences disclosed herein are also provided. Known methods can be used to isolate the corresponding genes using the provided cDNA sequences. These methods include preparation of probes or primers from the nucleotide sequences shown in SEQ ID NOs: 1, 2, 3, 4, 5, 6, 7, 8, 9, 10, 11, 12, 13, 14, 15, 16, 17, 18, and 19 for use in identifying or amplifying the genes from human genomic libraries or other sources of human genomic DNA.

The coding sequences shown in SEQ ID NOs: 1, 2, 3, 4, 5, 6, 7, 8, 9, 10, 11, 12, 13, 14, 15, 16, 17, 18, and 19 can be made using reverse transcriptase with human mRNA as a template. Amplification by PCR can also be used to obtain the polynucleotides, using either genomic DNA or cDNA as a template. Polynucleotide molecules of the invention can also be made using the techniques of synthetic chemistry given the sequences disclosed herein. The degeneracy of the genetic code permits alternate nucleotide sequences which will encode the amino acid sequences shown in SEQ ID Nos: 20, 21, 22, 23, 24, 25, 26, 27, 28, 29, 30, 31, 32, 33, 34, 35, 36, 37, and 38 to be synthesized. All such nucleotide sequences are within the scope of the present invention.

Polynucleotide molecules of the invention can be propagated in vectors and cell lines as is known in the art. Polynucleotide molecules can be on linear or circular molecules. They can be on autonomously replicating molecules or on molecules without replication sequences. For propagation, polynucleotides of the invention can be introduced into suitable host cells using any techniques available in the art, as described above.

Subgenomic polynucleotides of the invention can be used to propagate additional copies of the polynucleotides or to express protein, polypeptides, or fusion proteins. The subgenomic polynucleotides disclosed herein can also be used, for example, as biomarkers for tissues or chromosomes, as molecular weight markers for DNA gels, to elicit immune responses, such as the formation of antibodies against single- or double-stranded DNA, and in DNA-ligand interaction assays, to detect proteins or other molecules which interact with the nucleotide sequences.

Disease states may be associated with alterations in the expression of genes which encode proteins of the invention. Polynucleotide sequences disclosed herein can also be used to determine the involvement of any of these sequences in disease states. For example, a gene in a diseased cell can be sequenced and compared with a wild-type coding sequence of the invention. Alternatively, nucleotide probes can be constructed and used to detect normal or altered (mutant) forms of mRNA in a diseased cell. Subgenomic polynucleotides of the invention can also be used to design diagnostic tests and therapeutic compositions for diseases which may be associated with altered expression of these genes.

The present invention provides both full-length and mature forms of the disclosed proteins. Full-length forms of the proteins have the amino acid sequences shown in SEQ ID Nos: 20, 21, 22, 23, 24, 25, 26, 27, 28, 29, 30, 31, 32, 33, 34, 35, 36, 37, and 38. The full-length forms of a protein can be processed enzymatically to remove a signal sequence, resulting in a mature form of the protein. Signal sequences can be identified by examination of the amino acid sequences disclosed herein and comparison with amino acid sequences of known signal sequences (see, e.g., von Heijne, 1985; Kaiser & Botstein, 1986). Similarly, transmembrane domains can be identified by examination of the amino acid sequences disclosed herein. A transmembrane domain typically contains a long stretch of 15-30 hydrophobic amino acids.

Other domains with predicted functions can also be identified. For example, the protein having the amino acid sequence shown in SEQ ID NO: 23 comprises a Kunitz type serine protease inhibitor domain spanning amino acids 68 to 122 of SEQ ID NO: 23. The protein having the amino acid sequence shown in SEQ ID NO: 20 contains a zinc-finger motif.

Allelic variants of the disclosed subgenomic polynucleotides can occur and encode proteins which are identical, homologous, or substantially related to amino acid sequences disclosed herein (see below).

Allelic variants of subgenomic polynucleotides of the invention can be identified by hybridization of putative allelic variants with nucleotide sequences disclosed herein under stringent conditions. For example, by using the following wash conditions—2×SCC, 0.1% SDS, room temperature twice, 30 minutes each; then 2×SCC, 0.1% SDS, 50° C. once, 30 minutes; then 2×SCC, room temperature twice, 10 minutes each—allelic variants can be identified which contain at most about 25-30% basepair mismatches. More preferably, allelic variants contain 15-25% basepair mismatches, even more preferably 5-15% basepair mismatches.

Protein variants of secreted proteins of the invention are also included. Amino acids which are not involved in regions which determine biological activity can be deleted or modified without affecting biological function. Preferably, protein variants of the invention have amino acid sequences which are at least 85%, 90%, or 95% identical to the amino acid sequences disclosed herein and have similar biological properties (see below). More preferably, the molecules are 98% identical. Modifications of interest in the protein sequences can include the alteration, substitution, replacement, insertion or deletion of a selected amino acid residue. Proteins or derivatives can be either glycosylated or unglycosylated. Techniques for making such modifications are well known to those skilled in the art (see, e.g., U.S. Pat. No. 4,518,584). Alternatively, variants of proteins disclosed herein can be constructed using techniques of synthetic chemistry or using recombinant DNA methods.

Preferably, amino acid changes in variants or derivatives of proteins of the invention are conservative amino acid changes, i.e., substitutions of similarly charged or uncharged amino acids. A conservative amino acid change involves substitution of one amino acid for another amino acid of a family of amino acids which are structurally related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified as aromatic amino acids. It is reasonable to expect that an isolated replacement of a leucine with an isoleucine or valine, an aspartate with a glutamate, a threonine with a serine, or a similar replacement of an amino acid with a structurally related amino acid will not have a major effect on the binding properties of the resulting molecule, especially if the replacement does not involve an amino acid at a binding site involved in an interaction of the protein. Non-naturally occurring amino acids can also be used to form protein variants of the invention.

Whether an amino acid change results in a functional protein or polypeptide can readily be determined by assaying biological properties of the disclosed proteins or polypeptides, as described below. Species homologs of human subgenomic polynucleotides and proteins of the invention can also be identified by making suitable probes or primers and screening cDNA expression libraries from other species, such as mice, monkeys, yeast, or bacteria.

In the case of proteins which are membrane-bound, such as cell surface receptor proteins, soluble forms of the proteins can be obtained by deleting the nucleotide sequences which encode part or all of the intracellular and transmembrane domains of the protein and expressing a fully secreted form of the protein in a host cell. Techniques for identifying intracellular and transmembrane domains, such as homology searches, can be used to identify such domains in proteins of the invention using amino acid and nucleotide sequences disclosed herein.

Polypeptides consisting of less than full-length proteins of the present invention are also provided. Polypeptides of the invention can be linear or can be cyclized, for example, as described in Saragovi et al., 1992, Bio/Technology 10, 773-778 and McDowell et al., 1992, J. Amer. Chem. Soc. 114, 9245-9253. Polypeptides can be used, for example, as immunogens, diagnostic aids, or therapeutics, and to create fusion proteins, as described below.

Polypeptide molecules consisting of less than the entire amino acid sequences shown in SEQ ID Nos: 20, 21, 22, 23, 24, 25, 26, 27, 28, 29, 30, 31, 32, 33, 34, 35, 36, 37, and 38 are also provided. Such polypeptides comprise at least 6, 8, 10, 12, 15, 18, or 20 contiguous amino acids of an amino acid sequence shown in SEQ ID Nos: 20, 21, 22, 23, 24, 25, 26, 27, 28, 29, 30, 31, 32, 33, 34, 35, 36, 37, and 38. Polypeptide molecules of the invention can also possess minor amino acid alterations which do not substantially affect the ability of the polypeptides to interact with specific molecules, such as antibodies.

Derivatives of the polypeptides, such as glycosylated forms, aggregative conjugates with other molecules, and covalent conjugates with unrelated chemical moieties, are also provided. Derivatives also include allelic variants, species variants, and muteins. Covalent derivatives are prepared by linkage of functionalities to groups which are found in the amino acid chain or at the N- or C-terminal residue by means known in the art. Truncations or deletions of regions which do not affect biological function are also encompassed. Truncated or deleted polypeptides can be prepared synthetically or recombinantly, or by proteolytic digestion of purified or partially purified secreted proteins of the invention.

Fusion proteins comprising at least 6, 8, 10, 12, 15, 18, or 20 contiguous amino acids of the disclosed proteins can also be constructed. Human fusion proteins are useful, inter alia, for generating antibodies against amino acid sequences and for use in various assay systems. For example, fusion proteins can be used to identify proteins which interact with secreted proteins of the invention and influence their function. Physical methods, such as protein affinity chromatography, or library-based assays for protein-protein interactions, such as the yeast two-hybrid or phage display systems, can be used for this purpose. Such methods are well known in the art and can also be used as drug screens. Fusion proteins can also be used to target molecules to a specific location in a cell or to cause a molecule to be secreted or to be anchored in a cellular membrane.

Fusion proteins of the invention comprise two protein segments which are fused together with a peptide bond. The first protein segment comprises at least 6, 8, 10, 12, 15, 18, or 20 contiguous amino acids selected from an amino acid sequence shown in SEQ ID Nos: 20, 21, 22, 23, 24, 25, 26, 27, 28, 29, 30, 31, 32, 33, 34, 35, 36, 37, and 38. The first protein segment can also be a full-length protein (comprising a signal sequence) or a mature protein (lacking a signal sequence). The second protein segment can be a full-length protein or a protein fragment. The second protein or protein fragment can be labeled with a detectable marker, such as a radioactive, chemiluminescent, biotinylated, or fluorescent tag, or can be an enzyme which will generate a detectable product. Enzymes suitable for this purpose, such as β-galactosidase, are well known in the art.

Techniques for making fusion proteins, either recombinantly or by covalently linking two protein segments, are well known in the art. Fusion proteins comprising amino acid sequences of the invention can also be constructed, for example, using standard recombinant DNA methods to make a DNA construct which comprises contiguous nucleotides selected from SEQ ID NOs: 1, 2, 3, 4, 5, 6, 7, 8, 9, 10, 11, 12, 13, 14, 15, 16, 17, 18, and 19 and encoding the desired amino acids in proper reading frame with nucleotides encoding the second protein segment.

Proteins or polypeptides of the invention can be purified free from other components with which they are normally associated in a cell, such as carbohydrates, lipids, subcellular organelles, or other proteins. An isolated protein or polypeptide is at least 90% pure. Preferably, the preparations are 95% or 99% pure. The purity of a preparation can be assessed, for example, by examining electrophoretograms of protein or polypeptide preparations at several pH values and at several polyacrylamide concentrations, as is known in the art.

Standard biochemical methods can be used to isolate proteins of the invention from tissues which express the proteins or to isolate proteins, polypeptides, or fusion proteins from recombinant host cells into which a DNA construct has been introduced. Methods of protein purification, such as size exclusion chromatography, ammonium sulfate fractionation, ion exchange chromatography, affinity chromatography, crystallization, electrofocusing, or preparative gel electrophoresis, are well known and widely used in the art.

Alternatively, proteins, fusion proteins, or polypeptides of the invention can be produced by recombinant DNA methods or by synthetic chemical methods. Synthetic chemistry methods, such as solid phase peptide synthesis, can be used to synthesize proteins, fusion proteins, or polypeptides. For production of recombinant proteins, fusion proteins, or polypeptides, coding sequences selected from the nucleotide sequences shown in SEQ ID NOs: 1, 2, 3, 4, 5, 6, 7, 8, 9, 10, 11, 12, 13, 14, 15, 16, 17, 18, and 19 can be expressed in prokaryotic or eukaryotic host cells using expression systems known in the art. These expression systems include bacterial, yeast, insect, and mammalian cells (see below).

The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein, fusion protein, or polypeptide is typically greater than 95% pure. Further purification can be undertaken, using, for example, any of the techniques listed above. Proteins, fusion proteins, or polypeptides can also be tagged with an epitope, such as a “Flag” epitope (Kodak), and purified using an antibody which specifically binds to that epitope.

It may be necessary to modify a protein produced in yeast or bacteria, for example by phosphorylation or glycosylation of the appropriate sites, in order to obtain a functional protein. Such covalent attachments can be made using known chemical or enzymatic methods.

Proteins or polypeptides of the invention can also be expressed in cultured cells in a form which will facilitate purification. For example, a secreted protein or polypeptide can be expressed as a fusion protein comprising, for example, maltose binding protein, glutathione-S-transferase, or thioredoxin, and purified using a commercially available kit. Kits for expression and purification of such fusion proteins are available from companies such as New England BioLabs, Pharmacia, and Invitrogen.

The coding sequences disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Isolated proteins, polypeptides, or fusion proteins of the invention can be used to obtain a preparation of antibodies which specifically bind to epitopes comprising amino acid sequences of the invention. Antibodies of the invention can be used, for example, to detect proteins, polypeptides, or fusion proteins of the invention which are secreted into culture medium or to identify tissues or cells which express these molecules. The antibodies can be polyclonal or monoclonal or can be single chain antibodies. Techniques for raising polyclonal and monoclonal antibodies and for constructing single chain antibodies are well known in the art.

Antibodies of the invention bind specifically to epitopes comprising amino acid sequences of the invention, preferably to epitopes not present on other proteins. Typically a minimum number of contiguous amino acids to encode an epitope is 6, 8, or 10. However, more amino acids can be part of an epitope, for example, at least 15, 25, or 50, especially to form epitopes which involve non-contiguous residues. Specific binding antibodies do not detect other proteins on Western blots of proteins or in immunocytochemical assays. Specific binding antibodies provide a signal at least ten-fold lower than the signal provided with epitopes which do not comprise amino acid sequences of the invention. Antibodies which bind specifically to secreted proteins of the invention include those that bind to mature or full-length proteins, to polypeptides or degradation products, to fusion proteins, or to protein variants. In a preferred embodiment of the invention, the antibodies immunoprecipitate the desired protein, fusion protein, or polypeptide from solution and react with the protein, fusion protein, or polypeptide on Western blots of polyacrylamide gels.

Techniques for purifying antibodies are those which are available in the art. In a preferred embodiment, antibodies are affinity purified by passing the antibodies over a column to which amino acid sequences of the invention are bound. The bound antibody is then eluted, for example using a buffer with a high salt concentration. Any such technique may be chosen to purify antibodies of the invention.

The invention also provides DNA constructs, for expressing all or a portion of a protein of the invention in a host cell. The DNA construct comprises a promoter which is functional in the particular host cell selected. The skilled artisan can readily select an appropriate promoter from the large number of cell type-specific promoters known and used in the art. The DNA construct can also contain a transcription terminator which is functional in the host cell.

The expression construct comprises a polynucleotide segment which encodes all or a portion of a human protein encoded by SEQ ID NOs: 1, 2, 3, 4, 5, 6, 7, 8, 9, 10, 11, 12, 13, 14, 15, 16, 17, 18, and 19 or a variant thereof The polynucleotide segment is located downstream from the promoter. Transcription of the polynucleotide segment initiates at the promoter. DNA constructs can be linear or circular and can contain sequences, if desired, for autonomous replication.

The host cell comprising the DNA construct can be any suitable prokaryotic or eukaryotic cell. Expression systems in bacteria include those described in Chang et al., Nature (1978) 275: 615; Goeddel et al., Nature (1979) 281: 544; Goeddel et al., Nucleic Acids Res. (1980) 8: 4057; EP 36,776; U.S. Pat. No. 4,551,433; deBoer et al., Proc. Natl. Acad. Sci. USA (1983) 80: 21-25; and Siebenlist et al., Cell (1980) 20: 269.

Expression systems in yeast include those described in Hinnen et al., Proc. Natl. Acad. Sci. USA (1978) 75: 1929; Ito et al., J. Bacteriol. (1983) 153: 163; Kurtz et al., Mol. Cell. Biol. (1986) 6: 142; Kunze et al., J. Basic Microbiol.(1985) 25: 141; Gleeson et al., J. Gen. Microbiol. (1986) 132: 3459, Roggenkamp et al., Mol. Gen. Genet. (1986) 202 :302); Das et al., J. Bacteriol. (1984) 158: 1165; De Louvencourt et al., J. Bacteriol. (1983) 154: 737, Van den Berg et al., Bio/Technology (1990) 8: 135; Kunze et al, J. Basic Microbiol. (1985) 25: 141; Cregg et al., Mol. Cell. Biol. (1985) 5: 3376; U.S. Pat. No. 4,837,148; U.S. Pat. No. 4,929,555; Beach and Nurse, Nature (1981) 300: 706; Davidow et al., Curr. Genet. (1985) 10: 380; Gaillardin et al., Curr. Genet. (1985) 10: 49; Ballance et al., Biochem. Biophys. Res. Commun. (1983) 112: 284-289; Tilburn et al., Gene (1983) 26: 205-22;, Yelton et al., Proc. Natl. Acad. Sci. USA (1984) 81: 1470-1474; Kelly and Hynes, EMBO J. (1985) 4: 475479; EP 244,234; and WO 91/00357.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. (1988) 69: 765-776; Miller et al., Ann. Rev. Microbiol. (1988) 42: 177; Carbonell et al., Gene (1988) 73: 409; Maeda et al., Nature (1985) 315: 592-594; Lebacq-Verheyden et al., Mol. Cell. Biol. (1988) 8: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6: 47-55, Miller et al., in GENERIC ENGINEERING (Setlow, J. K. et al eds.), Vol. 8 (Plenum Publishing, 1986), pp. 277-279; and Maeda et al., Nature, (1985) 315: 592-594.

Mammalian expression can be accomplished as described in Dijkema et al., EMBO J. (1985) 4: 761; Gorman et al., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth. Enz. (1979) 58: 44; Barnes and Sato, Anal. Biochem. (1980) 102: 255; U.S. Pat. No. 4,767,704; U.S. Pat. No. 4,657,866; U.S. Pat. No. 4,927,762; U.S. Pat. No. 4,560,655; WO 90/103430, WO 87/00195, and U.S. RE 30,985.

DNA constructs of the invention can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, and calcium phosphate-mediated transfection.

Alternatively, expression of an endogenous gene encoding a protein of the invention can be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670, which is incorporated herein by reference.

The targeting sequence is a segment of at least 10, 12, 15, 20, or 50 contiguous nucleotides selected from the nucleotide sequences shown in SEQ ID NOs: 1, 2, 3, 4, 5, 6, 7, 8, 9, 10, 11, 12, 13, 14, 15, 16, 17, 18, and 19. The transcription unit is located upstream to a coding sequence of the endogenous gene. The exogenous regulatory sequence directs transcription of the coding sequence of the endogenous gene.

Secreted proteins of the invention have a variety of uses. For example, secreted proteins can be used in assays to determine biological activities, such as cytokine, cell proliferation, or cellular differentiation activities, tissue growth or regeneration, activin or inhibin activity, chemotactic or chemokinetic activity, hemostatic or thrombolytic activity, receptor/ligand activity, tumor inhibition, or anti-inflammatory activity. Assays for these activities are known in the art and are disclosed, for example, in U.S. Pat. No. 5,654,173, which is incorporated herein by reference.

Proteins of the invention can also be used as biomarkers, to identify tissues or cell types which express the proteins, or a stage- or disease-specific alteration in protein expression. Proteins of the invention can be used in protein interaction assays, to identify ligands or binding proteins. Compounds which affect the biological activities of the secreted proteins or their ability to interact with specific ligands can be identified using proteins of the invention in screening assays. Proteins and antibodies of the invention can also be used to design diagnostic tests and therapeutic compositions for diseases which may be associated with altered expression of these proteins. Fusion proteins comprising, for example, signal sequences or transmembrane domains of the disclosed proteins, can be used to target other protein domains to cellular locations in which the domains are not normally found, such as bound to a cellular membrane or secreted extracellularly.

Further objects, features, and advantages of the present invention will readily occur to the skilled artisan provided with the disclosure above.

### SYNOPSIS OF THE INVENTION

1. An isolated and purified human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

2. An isolated and purified human protein having an amino acid sequence which is at least 85% identical to an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

3. The isolated and purified human protein of item 2 wherein the amino acid sequence is at least 90% identical.

4. The isolated and purified human protein of item 2 wherein the amino acid sequence is at least 95% identical.

5. The isolated and purified human protein of item 2 wherein the amino acid sequence is at least 98% identical.

6. An isolated and purified human polypeptide comprising at least 6 contiguous amino acids of an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and

38.

7. A fusion protein comprising a first protein segment and a second protein segment fused together by means of a peptide bond, wherein the first protein segment consists of at least 6 contiguous amino acids selected from the group consisting of the amino acid sequences shown in SEQ ID Nos: 20, 21, 22, 23, 24, 25, 26, 27, 28, 29, 30, 31, 32, 33, 34, 35, 36, 37, and 38.

8. A preparation of antibodies which specifically bind to the human protein of item 1.

9. The preparation of antibodies of item 8 wherein the antibodies are monoclonal.

10. The preparation of antibodies of item 8 wherein the antibodies are polyclonal.

11. The preparation of antibodies of item 8 wherein the antibodies are single chain antibodies.

12. An isolated and purified subgenomic polynucleotide having a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

13. An isolated and purified subgenomic polynucleotide consisting of at least 10 contiguous nucleotides of a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

14. An isolated gene corresponding to a cDNA sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

15. A DNA construct for expressing all or a portion of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38, comprising:

a promoter; and

a polynucleotide segment encoding at least 6 contiguous amino acids of the human protein, wherein the polynucleotide segment is located downstream from the promoter, wherein transcription of the polynucleotide segment initiates at or 3′ to the promoter.

16. A host cell comprising a DNA construct comprising:

a promoter; and

a polynucleotide segment encoding at least 6 contiguous amino acids of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38, wherein the polynucleotide segment is located downstream from the pormoter and wherein transcription of the polynucleotide segment initiates at or 3′ to the promoter.

17. A homologously recombinant cell having incorporated therein a new transcription initiation unit, wherein the new transcription initiation unit comprises in 5′ to 3′ order:

(a) an exogenous regulatory sequence;

(b) an exogenous exon; and

(c) a splice donor site,

wherein the transcription initiation unit is located upstream to a coding sequence of a gene, wherein the gene comprises a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19, and wherein the exogenous regulatory sequence controls transcription of the coding sequence of the gene.

18. A method of producing a human protein, comprising the steps of:

growing a culture of a cell comprising a DNA construct comprising (1) a promoter and (2) a polynucleotide segment encoding at least 6 contiguous amino acids of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38, wherein the polynucleotide segment is located downstream from the promoter and wherein transcription of the polynucleotide segment initiates at or 3′ to the promoter; and;

purifying the protein from the culture.

19. A method of producing a human protein, comprising the steps of:

growing a culture of a homologously recombinant cell having incorporated therein a new transcription initiation unit, wherein the new transcription initiation unit comprises in 5′ to 3′ order:

(a) an exogenous regulatory sequence;

(b) an exogenous exon; and

(c) a splice donor site,

wherein the transcription initiation unit is located upstream to a coding sequence of a gene, wherein the gene comprises a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19 and wherein the exogenous regulatory sequence controls transcription of the coding sequence of the gene; and

purifying the protein from the culture.

20. A method of identifying a secreted polypeptide which is modified by rough microsomes, comprising the steps of:

transcribing in vitro a population of cDNA molecules whereby a population of cRNA molecules is formed;

translating a first portion of the population of cRNA molecules in vitro in the absence of rough microsomes whereby a first population of polypeptides is formed;

translating a second portion of the population of cRNA molecules in vitro in the presence of rough rnicrosomes whereby a second population of polypeptides is formed;

comparing the first population of polypeptides with the second population of polypeptides; and

detecting polypeptide members of the second population which have been modified by the rough microsomes.

21. The method of item 20 wherein the population of cDNA molecules is synthesized by reverse transcription of a population of mRNA molecules.

22. The method of item 21 wherein the mRNA molecules are isolated from a mammal.

23. The method of item 22 wherein the MRNA molecules are isolated from a human.

24. The method of item 20 wherein the population of cDNA molecules is obtained from a cDNA library.

25. The method of item 24 wherein the cDNA library is derived from a mammalian genome.

26. The method of item 25 wherein the CDNA library is derived from a human genome.

We claim:

1. An isolated and purified human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

2. An isolated and purified human protein having an amino acid sequence which is at least 85% identical to an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

3. An isolated and purified human polypeptide comprising at least 6 contiguous amino acids of an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38.

4. A fusion protein comprising a first protein segment and a second protein segment fused together by means of a peptide bond, wherein the first protein segment consists of at least 6 contiguous amino acids selected from the group consisting of the amino acid sequences shown in SEQ ID Nos: 20, 21, 22, 23, 24, 25, 26, 27, 28, 29, 30, 31, 32, 33, 34, 35, 36, 37, and 38.

5. A preparation of antibodies which specifically bind to the human protein of claim 1.

6. An isolated and purified subgenomic polynucleotide having a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

7. An isolated gene corresponding to a cDNA sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19.

8. A DNA construct for expressing all or a portion of a human protein having an amino acid sequence selected from the group consisting of the amino acid sequences shown in SEQ ID Nos: 20, 21, 22, 23, 24, 25, 26, 27, 28, 29, 30, 31, 32, 33, 34, 35, 36, 37, and 38, comprising:
a promoter; and
a polynucleotide segment encoding at least 6 contiguous amino acids of the human protein, wherein the polynucleotide segment is located downstream from the promoter, wherein transcription of the polynucleotide segment initiates at or 3′ to the promoter.

9. A host cell comprising a DNA construct comprising:
a promoter; and
a polynucleotide segment encoding at least 6 contiguous amino acids of a human protein having an amino acid sequence selected from the group consisting of the amino acid sequences shown in SEQ ID NOs: 20, 21, 22, 23, 24, 25, 26, 27, 28, 29, 30, 31, 32, 33, 34, 35, 36, 37, and 38, wherein the polynucleotide segment is located downstream from the promoter and wherein transcription of the polynucleotide segment initiates at or 3′ to the promoter.

10. A homologously recombinant cell having incorporated therein a new transcription initiation unit, wherein the new transcription initiation unit comprises in 5′ to 3′ order:
(a) an exogenous regulatory sequence;
(b) an exogenous exon; and
(c) a splice donor site,
wherein the transcription initiation unit is located upstream to a coding sequence of a gene, wherein the gene comprises a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19 and wherein the exogenous regulatory sequence controls transcription of the coding sequence of the gene.

11. A method of producing a human protein, comprising the steps of:
growing a culture of a cell comprising a DNA construct comprising (1) a promoter and (2) a polynucleotide segment encoding at least 6 contiguous amino acids of a human protein having an amino acid sequence selected from the group consisting of the amino acid sequences shown in SEQ ID NOs: 20, 21, 22, 23, 24, 25, 26, 27, 28, 29, 30, 31, 32, 33, 34, 35, 36, 37, and 38, wherein the polynucleotide segment is located downstream from the promoter and wherein transcription of the polynucleotide segment initiates at or 3′ to the promoter; and
purifying the protein from the culture.

12. A method of producing a human protein, comprising the steps of:
growing a culture of a homologously recombinant cell having incorporated therein a new transcription initiation unit, wherein the new transcription initiation unit comprises in 5′ to 3′ order:
(a) an exogenous regulatory sequence;
(b) an exogenous exon; and
(c) a splice donor site,
wherein the transcription initiation unit is located upstream to a coding sequence of a gene, wherein the gene comprises a nucleotide sequence selected from the group consisting of the nucleotide sequences shown in SEQ ID NOs: 1, 2, 3, 4, 5, 6, 7, 8, 9, 10, 11, 12, 13, 14, 15, 16, 17, 18, and 19 and wherein the exogenous regulatory sequence controls transcription of the coding sequence of the gene; and
purifying the protein from the culture.

13. A method of identifying a secreted polypeptide which is modified by rough microsomes, comprising the steps of:
transcribing in vitro a population of cDNA molecules whereby a population of cRNA molecules is formed;
translating a first portion of the population of cRNA molecules in vitro in the absence of rough microsomes whereby a first population of polypeptides is formed;
translating a second portion of the population of cRNA molecules in vitro in the presence of rough microsomes whereby a second population of polypeptides is formed;
comparing the first population of polypeptides with the second population of polypeptides; and
detecting polypeptide members of the second population which have been modified by the rough microsomes.


SEQUENCE LISTING (38 sequences)

SEQ ID NO: 1 - DNA, 2063 bp; single-stranded, linear
gaattcggcacgaggcctcagtcttccagggcggcggtgggtgtccgcttctctctgctcttcgactgcaccgcactcgcgcgtgaccctgactccccctagtcagctcagcggtgctgccatggcgtggcggcggcgcgaagccggcgtcggggctcgcggcgtgttggctctggcgttgctcgccctggccctgtgcgtgcccggggcccggggccgggctctcgagtggttctcggccgtggtaaacatcgagtacgtggacccgcagaccaacctgacggtgtggagcgtctcggagagtggccgcttcggcgacagctcgcccaaggagggcgcgcatggcctggtgggcgtcccgtgggcgcccggcggagacctcgagggctgcgcgcccgacacgcgcttcttcgtgcccgagcccggcggccgaggggccgcgccctgggtcgccctggtggctcgtgggggctgcaccttcaaggacaaggtgctggtggcggcgcggaggaacgcctcggccgtcgtcctctacaatgaggagcgctacgggaacatcaccttgcccatgtctcacgcgggaacaggaaatatagtggtcattatgattagctatccaaaaggaagagaaattttggagctggtgcaaaaaggaattccagtaacgatgaccataggggttggcacccggcatgtacaggagttcatcagcggtcagtctgtggtgtttgtggccattgccttcatcaccatgatgattatctcgttagcctggctaatattttactatatacagcgtttcctatatactggctctcagattggaagtcagagccatagaaaagaaactaagaaagttattggccagcttctacttcatactgtaaagcatggagaaaagggaattgatgttgatgctgaaaattgtgcagtgtgtattgaaaatttcaaagtaaaggatattattagaattctgccatgcaagcatatttttcatagaatatgcattgacccatggcttttggatcaccgaacatgtccaatgtgtaaacttgatgtcatcaaagccctaggatattggggagagcctggggatgtacaggagatgcctgctccagaatctcctcctggaagggatccagctgcaaatttgagtctagctttaccagatgatgacggaagtgatgacagcagtccaccatcagcctcccctgctgaatctgagccacagtgtgatcccagctttaaaggagatgcaggagaaaatacggcattgctagaagccggcaggagtgactctcggcatggaggacccatctcctagcacacgtgcccactgaagtggcaccaacagaagtttggcttgaactaaaggacattttattttttttactttagcacataatttgtatatttgaaaataatgtatattattttacctattagattctgatttgatatacaaaggactaagatattttcttcttgaagagacttttcgattagtcctcatatatttatctactaaaatagagtgtttaccatgaacagtgtgttgcttcagactattacaaagacaactggggcaggtactctaatataaaggacaggtggtgtttctaaataattggctgctatggttctgtaaaaaccagttaattctatttttcaaggtttttggcaaagcacatcaatgttagactagttgaagtggaattgtataattcaattcgataattgatctcatgggctttccctggaggaaaggttttttttgttgttttttttttaagaacttgaaacttgtaaactgagatgtctgtagcttttttgcccatctgtagtgtatgtgaagattctaaaacctgagagcactttttctttgtttagaattatgagaaaggcactagatgactttaggatttgcatttttccctttattgcctcatttcttgtgacgccttgttggggagggaaatctgtttattttttcctacaaataaaaagctaagattctatatcgcaaaaaaaaaaaaaaaaaaaaaaaaaattcctgcggccgc

SEQ ID NO: 2 - DNA, 1328 bp; single-stranded, linear
gaattcggcacgaggtaggcaagggataaaaaggcacctaaggcccttttgcaataagaagccagatggataaaggaagtgctggtcaccctggaggtgtactggtttggggaaggtccccggcccccacagccctctggggagcctcaccctggctctccccactcacctcagccctcaggcagcccctccacagggcccctctcctgcctggacagctctgctggtctccccgtcccctggagaagaacaaggccatgggtcggcccctgctgctgcccctgctgctcctgctgcagccgccagcatttctgcagcctggtggctccacaggatctggtccaagctacctttatggggtcactcaaccaaaacacctctcagcctccatgggtggctctgtggaaatccccttctccttctattacccctgggagttagccatagttcccaacgtgagaatatcctggagacggggccacttccacgggcagtccttctacagcacaaggccgccttccattcacaaggattatgtgaaccggctctttctgaactggacagagggtcaggagagcggcttcctcaggatctcaaacctgcggaaggaggaccagtctgtgtatttctgccgagtcgagctggacacccggagatcagggaggcagcagttgcagtccatcaaggggaccaaactcaccatcacccaggctgtcacaaccaccaccacctggaggcccagcagcacaaccaccatagccggcctcagggtcacagaaagcaaagggcactcagaatcatggcacctaagtctggacactgccatcagggttgcattggctgtcgctgtgctcaaaactgtcattttgggactgctgtgcctcctcctcctgtggtggaggagaaggaaaggtagcagggcgccaagcagtgacttctgaccaacagagtgtggggagaagggatgtgtattagccccggaggacgtgatgtgagacccgcttgtgagtcctccacactcgttccccattggcaagatacatggagagcaccctgaggacctttaaaaggcaaagccgcaaggcagaaggaggctgggtccctgaatcaccgactggaggagagttacctacaagagccttcatccaggagcatccacactgcaatgatataggaatgaggtctgaactccactgaattaaaccactggcatttgggggctgtttattatagcagtgcaaagagttcctttatcctccccaaggatggaaaaatacaatttattttgcttaccataaaaaaaaaaaaaaaaaaattcctgcggccgc

SEQ ID NO: 3 - DNA, 1689 bp; single-stranded, linear
gaattcggcacgagggcaagattcgatacaaaaccaatgaacctgtgtgggaggaaaacttcactttcttcattcacaatcccaagcgccaggaccttgaagttgaggtcagagacgagcagcaccagtgttccctggggaacctgaaggtccccctcagccagctgctcaccagtgaggacatgactgtgagccagcgcttccagctcagtaactcgggtccaaacagcaccatcaagatgaagattgccctgcgggtgctccatctcgaaaagcgagaaaggcctccagaccaccaacactcagctcaagtcaaacgtccctctgtgtccaaagaggggaggaaaacatccatcaaatctcatatgtctgggtctccaggccctggtggcagcaacacagctccatccacaccagtcattgggggcagtgataagcctggtatggaagaaaaggcccagccccctgaggccggccctcaggggctgcacgacctgggcagaagctcctccagcctcctggcctccccaggccacatctcagtcaaggagccgacccccagcatcgcctcggacatctcgctgcccatcgccacccaggagctgcggcaaaggctgaggcagctggaaaacgggacgaccctgggacagtctccactggggcagatccagctgaccatccggcacagctcgcagagaaacaagcttatcgtggtcgtgcatgcctgcagaaacctcattgccttctctgaagacggctctgacccctatgtccgcatgtatttattaccagacaagaggcggtcaggaaggaggaaaacacacgtgtcaaagaaaacattaaatccagtgtttgatcaaagctttgatttcagtgtttcgttaccagaagtgcagaggagaacgctcgacgttgccgtgaagaacagtggcggcttcctgtccaaagacaaagggctccttggcaaagtattggttgctctggcatctgaagaacttgccaaaggctggacccagtggtatgacctcacggaagatgggacgaggcctcaggcgatgacatagccgcagcaggcaggaggcgtcctcttcagcgtagctctccacctctacccggaacacaccctctcacagacgtaccaatgttatttttataatttcatggatttagttatacataccttaatagttttataaaattgttgacatttcaggcaaatttggccaatattatcattgaattttctgtgttggatttcctctaggatttcgccagttcctacaacgtgcagtagggcggcggtagctcttgtgtctgtggactctgctcagctgtgtccgtaggagtcggatgtgtctgtgctttattatggccttgtttatatatcactgaggtatactatgccatgtaaatagactattttttataatcttaacatgctggtttaaattcagaaggaaatagatcaaggaaatatatatattttcttctaaaacttattaaattcgtgtgacaaataatcattttcatcttggcagcaaaaagttctcagtgacctattttgtggtgtttctttttgaaaagaaaagctgaaatattattaaatgctagtatgtttctgcccattatgaaagatgaaataaagtattcaaaatattaaaaaaaaaaaaaaaaaattcctgcggccgc

SEQ ID NO: 4 - DNA, 1505 bp; single-stranded, linear
gaattcggcacgaggagcagatctgcaagagtttcgtttatggaggctgcttgggcaacaagaacaactaccttcgggaagaagagtgcattctagcctgtcggggtgtgcaaggtgggcctttgagaggcagctctggggctcaggcgactttcccccagggcccctccatggaaaggcgccatccagtgtgctctggcacctgtcagcccacccagttccgctgcagcaatggctgctgcatcgacagtttcctggagtgtgacgacacccccaactgccccgacgcctccgacgaggctgcctgtgaaaaatacacgagtggctttgacgagctccagcgcatccatttccccagcgacaaagggcactgcgtggacctgccagacacaggactctgcaaggagagcatcccgcgctggtactacaaccccttcagcgaacactgcgcccgctttacctatggtggttgttacggcaacaagaacaactttgaggaagagcagcagtgcctcgagtcttgtcgcggcatctccaagaaggatgtgtttggcctgaggcgggaaatccccattcccagcacaggctctgtggagatggctgtcgcagtgttcctggtcatctgcattgtggtggtggtagccatcttgggttactgcttcttcaagaaccagagaaaggacttccacggacaccaccaccacccaccacccacccctgccagctccactgtctccactaccgaggacacggagcacctggtctataaccacaccacgcggcccctctgagcctgggtctcaccggctctcacctggccctgcttcctgcttgccaaggcagaggcctgggctgggaaaaactttggaaccagactcttgcctgtttcccaggcccactgtgcctcagagaccagggctccagcccctcttggagaagtctcagctaagctcacgtcctgagaaagctcaaaggtttggaaggagcagaaaacccttgggccagaagtaccagactagatggacctgcctgcataggagtttggaggaagttggagttttgtttcctctgttcaaagctgcctgtccctaccccatggtgctaggaagaggagtggggtggtgtcagaccctggaggccccaaccctgtcctcccgagctcctcttccatgctgtgcgcccagggctgggaggaaggacttccctgtgtagtttgtgctgtaaagagttgctttttgtttatttaatgctgtggcatgggtgaagaggaggggaagaggcctgtttggcctctctatcctctcttcctcttcccccaagattgagctctctgcccttgatcagccccaccctggcctagaccagcagacagagccaggagaagctcagctgcattccgcagcccccacccccaaggttctccaacatcacagcccagcccgcccactgggtaataaaagtggtttgtggaaaaaaaaaaaaaaaaaaaaaagtcctgcggccgc

SEQ ID NO: 5 - DNA, 2002 bp; single-stranded, linear
gaattcggcacgagggccatggccgggctatcccgcgggtccgcgcgcgcactgctcgccgccctgctggcgtcgacgctgttggcgctgctcgtgtcgcccgcgcggggtcgcggcggccgggaccacggggactgggacgaggcctcccggctgccgccgctaccaccccgcgaggacgcggcgcgcgtggcccgcttcgtgacgcacgtctccgactggggcgctctggccaccatctccacgctggaggcggtgcgcggccggcccttcgccgacgtcctctcgctcagcgacgggcccccgggcgcgggcagcggcgtgccctatttctacctgagcccgctgcagctctccgtgagcaacctgcaggagaatccatatgctacactgaccatgactttggcacagaccaacttctgcaagaaacatggatttgatccacaaagtcccctttgtgttcacataatgctgtcaggaactgtgaccaaggtgaatgaaacagaaatggatattgcaaagcattcgttattcattcgacaccctgagatgaaaacctggccttccagccataattggttctttgctaagttgaatataaccaatatctgggtcctggactactttggtggaccaaaaatcgtgacaccagaagaatattataatgtcacagttcagtgaagcagactgtggtgaatttagcaacacttatgaagtttcttaaagtggctcatacacacttaaaaggcttaatgtttctctggaaagcgtcccagaatattagccagttttctgtcacatgctggtttgtttgcttgcttgtttacttgcttgtttaccaatagagttgacctgttattggatttcctggaagatgtggtagctacttttttcctattttgaagccattttcgtagagaaatatccttcactataatcaaataagttttgtcccatcaattccaaagatgtttccagtggtgctcttgaagaggaatgagtaccagttttaaattgcccattggcatttgaaggtagttgagtatgtgttctttattcctagaagccactgtgcttggtagagtgcatcactcaccacagctgcctcttgagctgcctgagcctggtgcaaaaggattggcccccattatggtgcttctgaataaatcttgccaagatagacaaacaatgatgaaactcagatggagcttcctactcatgttgatttatgtctcacaatcctgggtattgttaattcaacatagggtgaaactatttctgataaagaacttttgaaaaactttttatactctaaagtgatactcagaacaaaagaaagtcataaaactcctgaatttaatttccccacctaagtcgagacagtattatcaaaacacatgtgcacacagattattttttggctccaaaactggattgcaaaagaaagaggagagatattttgtgtgttcctggtattcttttataagtaaagttacccaggcatggaccagcttcagccagggacaaaatcccctcccaaaccactctccacagctttttaaaaatacttctactcttaacaattacctaaggttccttcaaacccccccaactcttaatagcttctagtgctgctacaatctaagtcaggtcaccagagggaagagaacatggcattaaaagaatcacatcttcagaagagaagacactaatattattacccatatacatgatttcagaagatgacataagattcctcttaaagaggaaatgtcaggaatcaagccactgaatccttaaagagaaaagttgaatatgagtcattgtgtctgaaaactgcaaagtgaacttaactgagatccagcaaacaggttctgtttaagaaaaataatttatactaaatttagtaaaatggacttcttattcaaagcatcaataattaaaagaattattttaaaaaaaaaaaaaaaaaaaaaaaaaaaaaaaaaattcctgcggccgc

SEQ ID NO: 6 - DNA, 1322 bp; single-stranded, linear
gaattcggcacgagggccacgactctgctggcatttcttctatagccactggaatctgatcctgattgtcttccactactaccaggccatcaccactccgcctgggtacccaccccagggcaggaatgatatcgccaccgtctccatctgtaagaagtgcatttaccccaagccagcccgaacacaccactgcagcatctgcaacaggtgtgtgctgaagatggatcaccactgcccctggctaaacaattgtgtgggccactataaccatcggtacttcttctctttctgctttttcatgactctgggctgtgtctactgcagctatggaagttgggaccttttccgggaggcttatgctgccattgagaaaatgaaacagctcgacaagaacaaactacaggcggttgccaaccagacttatcaccagaccccaccacccaccttctcctttcgagaaaggatgactcacaagagtcttgtctacctctggttcctgtgcagttctgtggcacttgccctgggtgccctaactgtatggcatgctgttctcatcagtcgaggtgagactagcatcgaaaggcacatcaacaagaaggagagacgtcggctacaggccaagggcagagtatttaggaatccttacaactacggctgcttggacaactggaaggtattcctgggtgtggatacaggaaggcactggcttactcgggtgctcttaccttctactcacttgccccatgggaatggaatgagctgggagccccctccctgggtgactgctcactcagcctctgtgatggcagtgtgagctggactgtgtcagccacgactcgagcactcattctgctccctatgttatttcaagggcctccaagggcagcttttctcagaatccttgatcaaaaagagccagtgggcctgccttagggtaccatgcaggacaattcaaggaccagcctttttaccactgcagaagaaagacacaatgtggagaaatcttaggactgacatccctttactcaggcaaacagaagttccaaccccagactaggggtcaggcagctagctacctaccttgcccagtgctgacccggacctcctccaggatacagcactggagttggccaccacctcttctacttgctgtctgaaaaaacacctgactagtacagctgagatcttggcttctcaacagggcaaagataccaggcctgctgctgaggtcactgccacttctcacatgctgcttaagggagcacaaataaaggtattcgatttttaaaaaaaaaaaaaaaaaaaaaaattcctgcggccgc

SEQ ID NO: 7 - DNA, 1573 bp; single-stranded, linear
gaattcggcacgaggagcctgccttcatctaggatggctcctctgggcatgctgcttgggctgctgatggccgcctgcttcaccttctgcctcagtcatcagaacctgaaggagtttgccctgaccaacccagagaagagcagcaccaaagaaacagagagaaaagaaaccaaagccgaggaggagctggatgccgaagtcctggaggtgttccacccgacgcatgagtggcaggcccttcagccagggcaggctgtccctgcaggatcccacgtacggctgaatcttcagactggggaaagagaggcaaaactccaatatgaggacaagttccgaaataatttgaaaggcaaaaggctggatatcaacaccaacacctacacatctcaggatctcaagagtgcactggcaaaattcaaggagggggcagagatggagagttcaaaggaagacaaggcaaggcaggctgaggtaaagcggctcttccgccccattgaggaactgaagaaagactttgatgagctgaatgttgtcattgagactgacatgcagatcatggtacggctgatcaacaagttcaatagttccagctccagtttggaagagaagattgctgcgctctttgatcttgaatattatgtccatcagatggacaatgcgcaggacctgctttcctttggtggtcttcaagtggtgatcaatgggctgaacagcacagagcccctcgtgaaggagtatgctgcgtttgtgctgggcgctgccttttccagcaaccccaaggtccaggtggaggccatcgaagggggagccctgcagaagctgctggtcatcctggccacggagcagccgctcactgcaaagaagaaggtcctgtttgcactgtgctccctgctgcgccacttcccctatgcccagcggcagttcctgaagctcggggggctgcaggtcctgaggaccctggtgcaggagaagggcacggaggtgctcgccgtgcgcgtggtcacactgctctacgacctggtcacggagaagatgttcgccgaggaggaggctgagctgacccaggagatgtccccagagaagctgcagcagtatcgccaggtacacctcctgccaggcctgtgggaacagggctggtgcgagatcacggcccacctcctggcgctgcccgagcatgatgcccgtgagaaggtgctgcagacactgggcgtcctcctgaccacctgccgggaccgctaccgtcaggacccccagctcggcaggacactggccagcctgcaggctgagtaccaggtgctggccagcctggagctgcaggatggtgaggacgagggctacttccaggagctgctgggctctgtcaacagcttgctgaaggagctgagatgaggccccacaccaggactggactgggatgccgctagtgaggctgaggggtgccagcgtgggtgggcttctcaggcaggaggacatcttggcagtgctggcttggccattaaatggaaacctgaaggccaaaaaaaaaaaaaaaaaaaaaaaaaaaaaaaaaaaaaaaaaaaattcctgcggccgc

SEQ ID NO: 8 - DNA, 1185 bp; single-stranded, linear
gaattcggcacgagggggctttaagggacagctgagccggcaggtggcagatcagatgtggcaggctgggaaaagacaagcctccagggccttcagcttgtacgccaacatcgacatcctcagaccctactttgatgtggagcctgctcaggtgcgaagcaggctcctggagtccatgatccctatcaagatggtcaacttcccccagaaaattgcaggtgaactctatggacctctcatgctggtcttcactctggttgctatcctactccatgggatgaagacgtctgacactattatccgggagggcaccctgatgggcacagccattggcacctgcttcggctactggctgggagtctcatccttcatttacttccttgcctacctgtgcaacgcccagatcaccatgctgcagatgttggcactgctgggctatggcctctttgggcattgcattgtcctgttcatcacctataatatccacctccacgccctcttctacctcttctggctgttggtgggtggactgtccacactgcgcatggtagcagtgttggtgtctcggaccgtgggccccacacagcggctgctcctctgtggcaccctggctgccctacacatgctcttcctgctctatctgcattttgcctaccacaaagtggtagaggggatcctggacacactggagggccccaacatcccgcccatccagagggtccccagagacatccctgccatgctccctgctgctcggcttcccaccaccgtcctcaacgccacagccaaagctgttgcggtgaccctgcagtcacactgaccccacctgaaattcttggccagtcctctttcccgcagctgcagagaggaggaagactattaaaggacagtcctgatgacatgtttcgtagatggggtttgcagctgccactgagctgtagctgcgtaagtacctccttgatgcctgtcggcacttctgaaaggcacaaggccaagaactcctggccaggactgcaaggctctgcagccaatgcagaaaatgggtcagctcctttgagaacccctccccacctaccccttccttcctctttatctctcccacattgtcttgctaaatatagacttggtaattaaaatgttgattgaagtctggaaaaaaaaaaaaaaaaaaattcctgcggccgc

SEQ ID NO: 9 - DNA, 1226 bp; single-stranded, linear
gaattcggcacgaggcaagccaccatcttccttcggcctgcacccctttaaaggcacccagacccctctggaaaaagatgaactgaagccctttgacatcctccagcctaaggagtacttccagctcagccgccacacggtcattaagatgggaagtgagaacgaggccctggatctctccatgaagtcagtgccctggctcaaggctggtgaagtcagtcccccaatcttccaggaagatgcagccctagacctgtcagtggcagcccaccggaaatccgagcctccccctgagacactgtatgacagtggtgcatcagtggacagctcaggtcacacagtgatggagaaacttcccagtggcatggaaatttcttttgcccctgccacgtcccatgaggccccagccatgatggatagtcacatcagcagcagtgatgctgctaccgagatgctcagccagcccaaccaccccagcggcgaagtcaaggctgaaaataacattgagatggtgggcgagtcccaggcggccaaggtcattgtctctgtcgaagatgctgtgcctaccatattctgtggcaagatcaaaggcctctcaggggtgtccaccaaaaacttctccttcaaaagagaagactccgtgcttcagggctatgacatcaacagccaaggggaagagtccatgggaaatgcagagccccttaggaaacccatcaaaaaccggagcataaagttaaagaaagtgaactcccaggaagtacacatgctcccaatcaaaaaacaacggctggccaccttttttccaagaaagtaaataacggctttttaaaatttgtatgattataatatggggaaaggtgcattggttttataaaaaggcatttaaaacaaattatctttgttaattattttggggagtagttgggaaatggaaaggtgaattggctctagaggccctgtatgctagtatcattttcttttttaatttttgacttttcacaaatgagtaaataagagcaacctatttttcaagcagattgcacattttttgcagctttaatggaatattgggtgaattagaggggtaaaaaaagctattttcattgccacaaagtgctttgatgatgtaatacctaataaagggtaggatgaatatttcacaataaatgtttgtttgcactaaaaaaaaaaaaaaaaaaaaaaaaaaaaaaaattcctgcggccgc

SEQ ID NO: 10 - DNA, 1049 bp; single-stranded, linear
gaattcggcacgagggcgccatggtgaaggtgacgttcaactccgctctggcccagaaggaggccaagaaggacgagcccaagagcggcgaggaggcgctcatcatcccccccgacgccgtcgcggtggactgcaaggacccagatgatgtggtaccagttggccaaagaagagcctggtgttggtgcatgtgctttggactagcatttatgcttgcaggtgttattctaggaggagcatacttgtacaaatattttgcacttcaaccagatgacgtgtactactgtggaataaagtacatcaaagatgatgtcatcttaaatgagccctctgcagatgccccagctgctctctaccagacaattgaagaaaatattaaaatctttgaagaagaagaagttgaatttatcagtgtgcctgtcccagagtttgcagatagtgatcctgccaacattgttcatgactttaacaagaaacttacagcctatttagatcttaacctggataagtgctatgtgatccctctgaacacttccattgttatgccacccagaaacctactggagttacttattaacatcaaggctggaacctatttgcctcagtcctatctgattcatgagcacatggttattactgatcgcattgaaaacattgatcacctgggtttctttatttatcgactgtgtcatgacaaggaaacttacaaactgcaacgcagagaaactattaaaggtattcagaaacgtgaagccagcaattgtttcgcaattcggcattttgaaaacaaatttgccgtggaaactttaatttgttcttgaacagtcaagaaaaacattattgaggaaaattaatatcacagcataaccccaccctttacattttgttgcagttgattattttttaaagtcttctttcatgtaagtagcaaacagggctttactatcttttcatctcattaattcaattaaaaccattaccttaaaaaaaaaaaaaaaaaaaaaaaaaaaaaaaaaaaaaaaaaaaaaaaaaaaaattcctgcggccgc

SEQ ID NO: 11 - DNA, 1142 bp; single-stranded, linear
gaattcggcacgaggggagaatactttttgcgatgcctactggagactttgattcgaagcccagttgggccgaccaggtggaggaggagggggaggacgacaaatgtgtcaccagcgagctcctcaaggggatccctctggccacaggtgacaccagcccagagccagagctactgccgggagctccactgccgcctcccaaggaggtcatcaacggaaacataaagacagtgacagagtacaagatagatgaggatggcaagaagttcaagattgtccgcaccttcaggattgagacccggaaggcttcaaaggctgtcgcaaggaggaagaactggaagaagttcgggaactcagagtttgacccccccggacccaatgtggccaccaccactgtcagtgacgatgtctctatgacgttcatcaccagcaaagaggacctgaactgccaggaggaggaggaccctatgaacaaattcaagggccagaagatcgtgtcctgccgcatctgcaagggcgaccactggaccacccgctgcccctacaaggatacgctggggcccatgcagaaggagctggccgagcagctgggcctgtctactggcgagaaggagaagctgccgggagagctagagccggtgcaggccacgcagaacaagacagggaagtatgtgccgccgagcctgcgcgacggggccagccgccgcggggagtccatgcagcccaaccgcagagccgacgacaacgccaccatccgtgtcaccaacttgcgcagaggacacgcgtgagaccgacctgcaggagctcttccggcctttcggctccatctcccgcatctacctggctaaggacaagaccactggccaatccaagggctttgccttcatcagcttccaccgccgcgaggatgctgcgcgtgccattgccggggtgtccggctttggctacgaccacctcatcctcaacgtcgagtgggccaagccgtccaccaactaagccagctgccactgtgtactcggtccgggacccttggcgacagaagacagcctccgagagcgcgggctccaagggcaataaagcagctccactctcaaaaaaaaaaaaaaaaaaaaaaaaaaaaaaaaaaaaattcctgcggccgc

SEQ ID NO: 12 - DNA, 1696 bp; single-stranded, linear
gaattcggcacgagggaaacatggcggtaggctgggaccataacacaagcatgactatatgaaggaagaggaaggttttcctgaagatgaggcgactgaatcggaaaaaaactttaagtttggtaaaagagttggatgcctttccgaaggttcctgagagctatgtagagacttcagccagtggaggtacagtttctctaatagcatttacaactatggctttattaaccataatggaattctcagtatatcaagatacatggatgaagtatgaatacgaagtagacaaggatttttctagcaaattaagaattaatatagatattactgttgccatgaagtgtcaatatgttggagcggatgtattggatttagcagaaacaatggttgcatctgcagatggtttagtttatgaaccaacagtatttgatctttcaccacagcagaaagagtggcagaggatgctgcagctgattcagagtaggctacaagaagagcattcacttcaagatgtgatatttaaaagtgcttttaaaagtacatcaacagctcttccaccaagagaagatgattcatcacagtctccaaatgcatgcagaattcatggccatctatatgtcaataaagtagcagggaattttcacataacagtgggcaaggcaattccacatcctcgtggtcatgcacatttggcagcacttgtcaaccatgaatcttacaatttttctcatagaatagatcatttgtcttttggagagcttgttccagcaattattaatcctttagatggaactgaaaaaattgctatagatcacaaccagatgttccaatattttattacagttgtgccaacaaaactacatacatataaaatatcagcagacacccatcagttttctgtgacagaaagggaacgtatcattaaccatgctgcaggcagccatggagtctctgggatatttatgaaatatgatctcagttctcttatggtgacagttactgaggagcacatgccattctggcagttttttgtaagactctgtggtattgttggaggaatcttttcaacaacaggcatgttacatggaattggaaaatttatagttgaaataatttgctgtcgtttcagacttggatcctataaacctgtcaattctgttccttttgaggatggccacacagacaaccacttacctcttttagaaaataatacacattaacacctcccgattgaaggagaaaaactttttgcctgagacataaaacctttttttaataataaaatattgtgcaatatattcaaagaaaagaaaacacaaataagcagaaaacatacttattttaaaaaagaaaaaaaaggataaaaaaacccaaactgaaattctatatacgttgtgtctgttacaaatgtcgtagaagaaatcatgcagctaaacgatgaagaagcccaactggagtgttgctttgaagatgacgccttcttatattttcatagcaaatgggtggtatcaaaatcagacattgcttcttgctgataaaaagcctgaaggaaataagtgaaactacatctatgggaaaaaaaaaaacattgagaagtgcaaatgttcgcatccttttgtttttaaaagatatgatgtcagaataaaatgtggaaaacatacggaaaaaaaaaaaaaaaaaaaattcctgcggccgc

SEQ ID NO: 13 - DNA, 1100 bp; single-stranded, linear
gaattcggcacgaggcggcacgaggcggcacgagggtggcatatcacggccatggggtctcagcattccgctgctgctcgcccctcctcctgcaggcgaaagcaagaagatgacagggacggtttgctggctgaacgagagcaggaagaagccattgctcagttcccatatgtggaattcaccgggagagatagcatcacctgtctcacgtgccaggggacaggctacattccaacagagcaagtaaatgagttggtggctttgatcccacacagtgatcagagattgcgccctcagcgaactaagcaatatgtcctcctgtccatcctgctttgtctcctggcatctggtttggtggttttcttcctgtttccgcattcagtccttgtggatgatgacggcatcaaagtggtgaaagtcacatttaataagcaagactcccttgtaattctcaccatcatggccaccctgaaaatcaggaactccaacttctacacggtggcagtgaccagcctgtccagccagattcagtacatgaacacagtggtcagtacatatgtgactactaacgtctcccttattccacctcggagtgagcaactggtgaattttaccgggaaggccgagatgggaggaccgttttcctatgtgtacttcttctgcacggtacctgagatcctggtgcacaacatagtgatcttcatgcgaacttcagtgaagatttcatacattggcctcatgacccagagctccttggagacacatcactatgtggattgtggaggaaattccacagctatttaacaactgctattggttcttccacacagcgcctgtagaagagagcacagcatatgttcccaaggcctgagttctggacctacccccacgtggtgtaagcagaggaggaattggttcacttaactcccagcaaacatcctcctgccacttaggaggaaacacctccctatggtaccatttatgtttctcagaaccagcagaatcagtgcctagcctgtgcccagcaaatagttggcactcaataaagatttgcagaatttaaaaaaaaaaaaaaaaaaaaaaaaaattcctgcggccgc

SEQ ID NO: 14 - DNA, 1588 bp; single-stranded, linear
gaattcggcacgagggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaaccttttttttacagagcctaattaataacttaatgactgtgtacatagcaatgtgtgtgtatgtatatgtctgtgagctattaatgttattaattttcataaaagctggaaagcaaaaaaaaaaaaaaaaaattcctgcggccgc

SEQ ID NO: 15 - DNA, 1535 bp; single-stranded, linear
gaattcggcacgaggcggaagtcccgtctcacggttgccctggcagcgcgcgaggctggtgagtcggcagccctgtggcagccggcgggctggtttccatggttgcacgattaggaaccaccagctgctgcatcccatggccaggggtggcgtccaggtggcagagcagctaggaacgcaaggcctgaacctggggccagacaccctgctctcccggccatggtcaacgaccctccagtacctgccttactgtgggcccaggaggtgggccaagtcttggcaggccgtgcccgcaggctgctgctgcagtttggggtgctcttctgcaccatcctccttttgctctgggtgtctgtcttcctctatggctccttctactattcctatatgccgacagtcagccacctcagccctgtgcatttctactacaggaccgactgtgattcctccaccacctcactctgctccttccctgttgccaatgtctcgctgactaagggtggacgtgatcgggtgctgatgtatggacagccgtatcgtgttaccttagagcttgagctgccagagtcccctgtgaatcaagatttgggcatgttcttggtcaccatttcctgctacaccagaggtggccgaatcatctccacttcttcgcgttcggtgatgctgcattaccgctcagacctgctccagatgctggacacactggtcttctctagcctcctgctatttggctttgcagagcagaagcagctgctggaggtggaactctacgcagactatagagagaactcgtacgtgccgaccactggagcgatcattgagatccacagcaagcgcatccagctgtatggagcctacctccgcatccacgcgcacttcactgggctcagatacctgctatacaacttcccgatgacctgcgccttcataggtgttgccagcaacttcaccttcctcagcgtcatcgtgctcttcagctacatgcagtgggtgtgggggggcatctggccccgacaccgcttctctttgcaggttaacatccgaaaaagagacaattcccggaaggaagtccaacgaaggatctctgctcatcagccagggcctgaaggccaggaggagtcaactccgcaatcagatgttacagaggatggtgagagccctgaagatccctcagggacagaggtcagctgtccgaggaggagaaaccagatcagcagcccctgagcggagaagaggagctagagcctgaggccagtgatggttcaggctcctgggaagatgcagctttgctgacggaggccaacctgcctgctcctgctcctgcttctgcttctgcccctgtcctagagactctgggcagctctgaacctgctgggggtgctctccgacagcgccccacctgctctagttcctgaagaaaaggggcagactcctcacattccagcactttcccacctgactcctctcccctcgtttttccttcaataaactattttgtgtcaaaaaaaaaaaaaaaaaaaaattcctgcggccgc

SEQ ID NO: 16 - DNA, 1322 bp; single-stranded, linear
gaattcggcacgagggcgggcgctacgggcttgactcccccaaggccgaggtccgcggccaggtgctggcgccgctgcccctccacggagttgctgatcatctgggctgtgatccacaaacccggttctttgtccctcctaatatcaaacagtggattgccttgctgcagaggggaaactgcacgtttaaagagaaaatatcacgggccgctttccacaatgcagttgctgtagtcatctacaataataaatccaaagaggagccagttaccatgactcatccaggcactggagatattattgctgtcatgataacagaattgaggggtaaggatattttgagttatctggagaaaaacatctctgtacaaatgacaatagctgttggaactcgaatgccaccgaagaacttcagccgtggctctctagtcttcgtgtcaatatcctttattgttttgatgattatttcttcagcatggctcatattctacttcattcaaaagatcaggtacacaaatgcacgcgacaggaaccagcgtcgtctcggagatgcagccaagaaagccatcagtaaattgacaaccaggacagtaaagaagggtgacaaggaaactgacccagactttgatcattgtgcagtctgcatagagagctataagcagaatgatgtcgtccgaattctcccctgcaagcatgttttccacaaatcctgcgtggatccctggcttagtgaacattgtacctgtcctatgtgcaaacttaatatattgaaggccctgggaattgtgccgaatttgccatgtactgataacgtagcattcgatatggaaaggctcaccagaacccaagctgttaaccgaagatcagccctcggcgacctcgccggcgacaactcccttggccttgagccacttcgaacttcggggatctcacctcttcctcaggatggggagctcactccgagaacaggagaaatcaacattgcagtaacaaaagaatggtttattattgccagttttggcctcctcagtgccctcacactctgctacatgatcatcagagccacagctagcttgaatgctaatgaggtagaatggttttgaagaagaaaaaacctgctttctgactgattttgccttgaaggaaaaaagaacctatttttgtgcatcatttaccaatcatgccacacaagcatttatttttagtacattttattttttcataaaattgctaatgccaaagctttgtattaaaagaaataaataataaaataaaaaaaaaaaaaaaaaaaaaaaaaaaaaaaaaaaaattcctgcggccgc

SEQ ID NO: 17 - DNA, 1711 bp; single-stranded, linear
gaattcggcacgaggccctcccgcgctcccggggcgcgcgggccgcgcccccgacgccctacatatactcaggtgcgccccacctgtccgcccgcacctgctggctcacctccgagccacctctgctgcgcaccgcagcctcggacctacagcccaggatactttgggacttgccggcgctcagaaacgcgcccagacggcccctccaccttttgtttgcctagggtcgccgagagcgcccggagggaaccgcctggccttcggggaccaccaattttgtctggaaccaccctcccggcgtatcctactccctgtgccgcgaggccatcgcttcactggaggggtcgatttgtgtgtagtttggtgacaagatttgcattcacctggcccaaaccctttttgtctctttgggtgaccggaaaactccacctcaagttttcttttgtggggctgccccccaagtgtcgtttgttttactgtagggtctcccgcccggcgcccccagtgttttctgagggcggaaatggccaattcgggcctgcagttgctgggcttctccatggccctgctgggctgggtgggtctggtggcctgcaccgccatcccgcagtggcagatgagctcctatgcgggtgacaacatcatcacggcccaggccatgtacaaggggctgtggatggactgcgtcacgcagagcacggggatgatgagctgcaaaatgtacgactcggtgctcgccctgtccgcggccttgcaggccactcgagccctaatggtggtctccctggtgctgggcttcctggccatgtttgtggccacgatgggcatgaagtgcacgcgctgtgggggagacgacaaagtgaagaaggcccgtatagccatgggtggaggcataattttcatcgtggcaggtcttgccgccttggtagcttgctcctggtatggccatcagattgtcacagacttttataaccctttgatccctaccaacattaagtatgagtttggccctgccatctttattggctgggcagggtctgccctagtcatcctgggaggtgcactgctctcctgttcctgtcctgggaatgagagcaaggctgggtaccgtgcaccccgctcttaccctaagtccaactcttccaaggagtatgtgtgacctgggatctccttgccccagcctgacaggctatgggagtgtctagatgcctgaaagggcctggggctgagctcagcctgtgggcagggtgccggacaaaggcctcctggtcactctgtccctgcactccatgtatagtcctcttgggttgggggtgggggggtgccgttggtgggagagacaaaaagagggagagtgtgctttttgtacagtaataaaaaataagtattgggaagcaggcttttttcccttcagggcctctgctttcctcccgtccagatccttgcagggagcttggaaccttagtgcacctacttcagttcagaacacttagcaccccactgactccactgacaattgactaaaagatgcaggtgctcgtatctcgacattcattcccacccccctcttatttaaatagctaccaaagtacttcttttttaataaaaaaataaagatttttattaggtaaaaaaaaaaaaaaaaaaaaaaaaaaaaaaaaaaaaaaaaaaaaaaaaaaaaaaaaaaaaaaaaaaaaattcctgcggccgc

SEQ ID NO: 18 - DNA, 1553 bp; single-stranded, linear
gaattcggcacgagggcaggtccagagtaaagtcactgaagagtggaagcgaggaaggaacaggatgattagacctcagctgcggaccgcggggctgggacgatgcctcctgccggggctgctgctgctcctggtgcccgtcctctgggccggggctgaaaagctacatacccagccctcctgccccgcggtctgccagcccacgcgctgccccgcgctgcccacctgcgcgctggggaccacgccggtgttcgacctgtgccgctgttgccgcgtctgccccgcggccgagcgtgaagtctgcggcggggcgcagggccaaccgtgcgccccggggctgcagtgcctccagccgctgcgccccgggttccccagcacctgcggttgcccgacgctgggaggggccgtgtgcggcagcgacaggcgcacctaccccagcatgtgcgcgctccgggccgaaaaccgcgccgcgcgccgcctgggcaaggtcccggccgtgcctgtgcagtgggggaactgcggggatacagggaccagaagcgcaggcccgctcaggaggaattacaacttcatcgccgcggtggtggagaaggtggcgccatcggtggttcacgtgcagctgtggggcaggttacttcacggcagcaggcttgttcctgtgtacagtggctctgggttcatagtgtctgaggacgggctcattattaccaatgcccatgttgtcaggaaccagcagtggattgaggtggtgctccagaatggggcccgttatgaagctgttgtcaaggatattgaccttaaattggatcttgcggtgattaagattgaatcaaatgctgaacttcctgtactgatgctgggaagatcatctgaccttcgggctggagagtttgtggtggctttgggcagcccattttctctgcagaacacagctactgcaggaattgtcagcaccaaacagcgagggggcaaagaactggggatgaaggattcagatatggactacgtccagattgatgccacaattaactatgggaattctggtggtcctctggtgaacttggatggtgatgtgattggcgtcaattcattgagggtgactgatggaatctcctttgcaattccttcagatcgagttaggcagttcttggcagaataccatgagcaccagatgaaaggaaaggcgttttcaaataagaaatatctgggtctgcaaatgctgtccctcactgtgccccttagtgaagaattgaaaatgcattatccagatttccctgatgtgagttctggggtttatgtatgtaaagtggttgaaggaacagctgctcaaagctctggattgagagatcacgatgtaattgtcaacataaatgggaaacctattactactacaactgatgttgttaaagctcttgacagtgattccctttccatggctgttcttcggggaaaagataatttgctcctgacagtcatacctgaaacaatcaattaaatatcttgttttaaagtgggattatctaaaaaaaaaaaaaaaaaattcctgcggccgc

SEQ ID NO: 19 - DNA (cDNA), 1596 bp; single-stranded, linear
gaattcggcacgaggggagccgctcccggagcccggccgtagaggctgcaatcgcagccgggagcccgcagcccgcgccccgagcccgccgccgcccttcgagggcgccccaggccgcgccatggtgaaggtgacgttcaactccgctctggcccagaaggaggccaagaaggacgagcccgagagcggcgaggaggcgctcatcatcccccccgacgccgtcgcggtggactgcaaggacccagatgatgtggtaccagttggccaaagaagagcctggtgttggtgcatgtgctttggactagcatttatgcttgcaggtgttattctaggaggagcatacttgtacaaatattttgcacttcaaccagatgacgtgtactactgtggaataaagtacatcaaagatgatgtcatcttaaatgagccctctgcagatgccccagctgctctctaccagacaattgaagaaaatattaaaatctttgaagaagaagaagttgaatttatcagtgtgcctgtcccagagtttgcagatagtgatcctgccaacattgttcatgactttaacaagaaacttacagcctatttagatcttaacctggataagtgctatgtgatccctctgaacacttccattgttatgccacccagaaacctactggagttacttattaacatcaaggctggaacctatttgcctcagtcctatctgattcatgagcacatggttattactgatcgcattgaaaacattgatcacctgggtttctttatttatcgactgtgtcatgacaaggaaacttacaaactgcaacgcagagaaactattaaaggtattcagaaacgtgaagccagcaattgtttcgcaattcggcattttgaaaacaaatttgccgtggaaactttaatttgttcttgaacagtcaagaaaaacattattgaggaaaattaatatcacagcataaccccaccctttacattttgtgcagtgatattttttaaagtctctttcatgtaagtagcaaacagggctttactatcttttcatctcattaattcaattaaaaccattaccttaaaatttttttctttcgaagtgtggtgtcttttatatttgaattagtaactgtatgaagtcatagataatagtacatgtcaccttaggtagtaggaagaattacaatttctttaaatcatttatctggatttttatgttttattagcattttcaagaagacggattatctagagaataatcatatatatgcatacgtaaaaatggaccacagtgacttatttgtagttgttagttgccctgctacctagtttgttagtgcatttgagcacacattttaattttcctctaattaaaatgtgcagtattttcagtgtcaaatatatttaactatttagagaatgatttccacctttatgttttaatatcctaggcatctgctgtaataatattttagaaaatgtttggaatttaagaaataacttgtgttactaatttgtataacccatatctgtgcaatggaatataaatatcacaaagttgtttaaaaaaaaaaaaaaaaaaattcctgcggccgc

SEQ ID NO: 20 - protein, 400 aa
MAWRRREAGVGARGVLALALLALALCVPGARGRALEWFSAVVNIEYVDPQTNLTVWSVSESGRFGDSSPKEGAHGLVGVPWAPGGDLEGCAPDTRFFVPEPGGRGAAPWVALVARGGCTFKDKVLVAARRNASAVVLYNEERYGNITLPMSHAGTGNIVVIMISYPKGREILELVQKGIPVTMTIGVGTRHVQEFISGQSVVFVAIAFITMMIISLAWLIFYYIQRFLYTGSQIGSQSHRKETKKVIGQLLLHTVKHGEKGIDVDAENCAVCIENFKVKDIIRILPCKHIFHRICIDPWLLDHRTCPMCKLDVIKALGYWGEPGDVQEMPAPESPPGRDPAANLSLALPDDDGSDDSSPPSASPAESEPQCDPSFKGDAGENTALLEAGRSDSRHGGPIS

SEQ ID NO: 21 - protein, 291 aa
MDKGSAGHPGGVLVWGRSPAPTALWGASPWLSPLTSALRQPLHRAPLLPGQLCWSPRPLEKNKAMGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 22 - protein, 293 aa
MTVSQRFQLSNSGPNSTIKMKIALRVLHLEKRERPPDHQHSAQVKRPSVSKEGRKTSIKSHMSGSPGPGGSNTAPSTPVIGGSDKPGMEEKAQPPEAGPQGLHDLGRSSSSLLASPGHISVKEPTPSIASDISLPIATQELRQRLRQLENGTTLGQSPLGQIQLTIRHSSQRNKLIVVVHACRNLIAFSEDGSDPYVRMYLLPDKRRSGRRKTHVSKKTLNPVFDQSFDFSVSLPEVQRRTLDVAVKNSGGFLSKDKGLLGKVLVALASEELAKGWTQWYDLTEDGTRPQAMT

SEQ ID NO: 23 - protein, 206 aa [sequence omitted]

SEQ ID NO: 24 - protein, 220 aa
MAGLSRGSARALLAALLASTLLALLVSPARGRGGRDHGDWDEASRLPPLPPREDAARVARFVTHVSDWGALATISTLEAVRGRPFADVLSLSDGPPGAGSGVPYFYLSPLQLSVSNLQENPYATLTMTLAQTNFCKKHGFDPQSPLCVHIMLSGTVTKVNETEMDIAKHSLFIRHPEMKTWPSSHNWFFAKLNITNIWVLDYFGGPKIVTPEEYYNVTVQ

SEQ ID NO: 25 - protein, 197 aa
MDHHCPWLNNCVGHYNHRYFFSFCFFMTLGCVYCSYGSWDLFREAYAAIEKMKQLDKNKLQAVANQTYHQTPPPTFSFRERMTHKSLVYLWFLCSSVALALGALTVWHAVLISRGETSIERHINKKERRRLQAKGRVFRNPYNYGCLDNWKVFLGVDTGRHWLTRVLLPSTHLPHGNGMSWEPPPWVTAHSASVMAV

SEQ ID NO: 26 - protein, 451 aa
MAPLGMLLGLLMAACFTFCLSHQNLKEFALTNPEKSSTKETERKETKAEEELDAEVLEVFHPTHEWQALQPGQAVPAGSHVRLNLQTGEREAKLQYEDKFRNNLKGKRLDINTNTYTSQDLKSALAKFKEGAEMESSKEDKARQAEVKRLFRPIEELKKDFDELNVVIETDMQIMVRLINKFNSSSSSLEEKIAALFDLEYYVHQMDNAQDLLSFGGLQVVINGLNSTEPLVKEYAAFVLGAAFSSNPKVQVEAIEGGALQKLLVILATEQPLTAKKKVLFALCSLLRHFPYAQRQFLKLGGLQVLRTLVQEKGTEVLAVRVVTLLYDLVTEKMFAEEEAELTQEMSPEKLQQYRQVHLLPGLWEQGWCEITAHLLALPEHDAREKVLQTLGVLLTTCRDRYRQDPQLGRTLASLQAEYQVLASLELQDGEDEGYFQELLGSVNSLLKELR

SEQ ID NO: 27 - protein, 254 aa [sequence omitted]

SEQ ID NO: 28 - protein, 221 aa
MGSENEALDLSMKSVPWLKAGEVSPPIFQEDAALDLSVAAHRKSEPPPETLYDSGASVDSSGHTVMEKLPSGMEISFAPATSHEAPAMMDSHISSSDAATEMLSQPNHPSGEVKAENNIEMVGESQAAKVIVSVEDAVPTIFCGKIKGLSGVSTKNFSFKREDSVLQGYDINSQGEESMGNAEPLRKPIKNRSIKLKKVNSQEVHMLPIKKQRLATFFPRK

SEQ ID NO: 29 - protein, 266 aa
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 30 - protein, 251 aa
MPTGDFDSKPSWADQVEEEGEDDKCVTSELLKGIPLATGDTSPEPELLPGAPLPPPKEVINGNIKTVTEYKIDEDGKKFKIVRTFRIETRKASKAVARRKNWKKFGNSEFDPPGPNVATTTVSDDVSMTFITSKEDLNCQEEEDPMNKFKGQKIVSCRICKGDHWTTRCPYKDTLGPMQKELAEQLGLSTGEKEKLPGELEPVQATQNKTGKYVPPSLRDGASRRGESMQPNRRADDNATIRVTNLRRGHA

SEQ ID NO: 31 - protein, 377 aa
MRRLNRKKTLSLVKELDAFPKVPESYVETSASGGTVSLIAFTTMALLTIMEFSVYQDTWMKYEYEVDKDFSSKLRINIDITVAMKCQYVGADVLDLAETMVASADGLVYEPTVFDLSPQQKEWQRMLQLIQSRLQEEHSLQDVIFKSAFKSTSTALPPREDDSSQSPNACRIHGHLYVNKVAGNFHITVGKAIPHPRGHAHLAALVNHESYNFSHRIDHLSFGELVPAIINPLDGTEKIAIDHNQMFQYFITVVPTKLHTYKISADTHQFSVTERERIINHAAGSHGVSGIFMKYDLSSLMVTVTEEHMPFWQFFVRLCGIVGGIFSTTGMLHGIGKFIVEIICCRFRLGSYKPVNSVPFEDGHTDNHLPLLENNTH

SEQ ID NO: 32 - protein, 250 aa
MGSQHSAAARPSSCRRKQEDDRDGLLAEREQEEAIAQFPYVEFTGRDSITCLTCQGTGYIPTEQVNELVALIPHSDQRLRPQRTKQYVLLSILLCLLASGLVVFFLFPHSVLVDDDGIKVVKVTFNKQDSLVILTIMATLKIRNSNFYTVAVTSLSSQIQYMNTVVSTYVTTNVSLIPPRSEQLVNFTGKAEMGGPFSYVYFFCTVPEILVHNIVIFMRTSVKISYIGLMTQSSLETHHYVDCGGNSTAI

SEQ ID NO: 33 - protein, 374 aa
MVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 34 - protein, 334 aa
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVTLELELPESPVNQDLGMFLVTISCYTRGGRIISTSSRSVMLHYRSDLLQMLDTLVFSSLLLFGFAEQKQLLEVELYADYRENSYVPTTGAIIEIHSKRIQLYGAYLRIHAHFTGLRYLLYNFPMTCAFIGVASNFTFLSVIVLFSYMQWVWGGIWPRHRFSLQVNIRKRDNSRKEVQRRISAHQPGPEGQEESTPQSDVTEDGESPEDPSGTEVSCPRRRNQISSP

SEQ ID NO: 35 - protein, 276 aa
MTHPGTGDIIAVMITELRGKDILSYLEKNISVQMTIAVGTRMPPKNFSRGSLVFVSISFIVLMIISSAWLIFYFIQKIRYTNARDRNQRRLGDAAKKAISKLTTRTVKKGDKETDPDFDHCAVCIESYKQNDVVRILPCKHVFHKSCVDPWLSEHCTCPMCKLNILKALGIVPNLPCTDNVAFDMERLTRTQAVNRRSALGDLAGDNSLGLEPLRTSGISPLPQDGELTPRTGEINIAVTKEWFIIASFGLLSALTLCYMIIRATASLNANEVEWF

SEQ ID NO: 36 - protein, 210 aa
MANSGLQLLGFSMALLGWVGLVACTAIPQWQMSSYAGDNIITAQAMYKGLWMDCVTQSTGMMSCKMYDSVLALSAALQATRALMVVSLVLGFLAMFVATMGMKCTRCGGDDKVKKARIAMGGGIIFIVAGLAALVACSWYGHQIVTDFYNPLIPTNIKYEFGPAIFIGWAGSALVILGGALLSCSCPGNESKAGYRAPRSYPKSNSSKEY

SEQ ID NO: 37 - protein, 476 aa
MIRPQLRTAGLGRCLLPGLLLLLVPVLWAGAEKLHTQPSCPAVCQPTRCPALPTCALGTTPVFDLCRCCRVCPAAEREVCGGAQGQPCAPGLQCLQPLRPGFPSTCGCPTLGGAVCGSDRRTYPSMCALRAENRAARRLGKVPAVPVQWGNCGDTGTRSAGPLRRNYNFIAAVVEKVAPSVVHVQLWGRLLHGSRLVPVYSGSGFIVSEDGLIITNAHVVRNQQWIEVVLQNGARYEAVVKDIDLKLDLAVIKIESNAELPVLMLGRSSDLRAGEFVVALGSPFSLQNTATAGIVSTKQRGGKELGMKDSDMDYVQIDATINYGNSGGPLVNLDGDVIGVNSLRVTDGISFAIPSDRVRQFLAEYHEHQMKGKAFSNKKYLGLQMLSLTVPLSEELKMHYPDFPDVSSGVYVCKVVEGTAAQSSGLRDHDVIVNINGKPITTTTDVVKALDSDSLSMAVLRGKDNLLLTVIPETIN

SEQ ID NO: 38 - protein, 266 aa
MVKVTFNSALAQKEAKKDEPESGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS